(19) United States
(12) Patent Application Publication
(10) Pub. No.: US 2004/0133242 A1
(43) Pub. Date: Jul. 8, 2004

(54) Medical device communication

(76) Inventors: Fred W. Chapman, Renton, WA (US); Mark P. Moore, Redmond, WA (US); Ward Silver, Vashon, WA (US)

Correspondence Address: SHUMAKER & SIEFFERT, P. A., 8425 SEASONS PARKWAY, SUITE 105, ST. PAUL, MN, 55125, US

(21) Appl. No.: 10/336,655

(22) Filed: Jan. 2, 2003

(51) Int. Cl. A61N 1/39

(52) U.S. Cl. 607/5

(57) ABSTRACT

Techniques are described for communication between medical devices that are used to monitor or treat a patient, such as defibrillators or patient monitors. The devices may engage in one-way or two-way communication, using the electrical conduction of the body of the patient to carry information. When the devices are capable of two-way communication, the first medical device may relay information to the second medical device through the body of the patient. The information relayed may pertain to the condition of the patient or the therapy administered.

### TECHNICAL FIELD

The invention relates to medical devices, and more particularly, to external defibrillators.

### BACKGROUND

A defibrillator is a device that stores energy, typically in one or more high-voltage capacitors, and delivers the stored energy to a patient. In particular, a defibrillator delivers energy to a heart that is undergoing fibrillation and has lost its ability to contract. Ventricular fibrillation is particularly life threatening because activity within the ventricles of the heart is so uncoordinated that virtually no pumping of blood takes place. An electrical pulse delivered to a fibrillating heart may depolarize the heart and cause it to reestablish a normal sinus rhythm.

An external defibrillator applies a defibrillation pulse via electrodes to generate a record of the monitoring and treatment of the patient placed upon the patient's chest. When a switch is closed, the defibrillator delivers at least some of the stored energy to the patient's chest. In some cases, a patient may need multiple shocks, and different quantities of energy may be delivered with each shock.

In some cases, the patient may be treated with more than one medical device. In a typical scenario, the patient may have suffered heart trouble in a public venue, such as an airport or a sports arena. The patient may have received prompt defibrillation therapy with an automated external defibrillators (AED). AEDs are available in many public venues, and can be brought to the patient quickly. Most AEDs are designed so that a minimally-trained operator, such as a security guard or a police officer, can use the AED to deliver therapy to the patient.

In this typical scenario, the AED is not the only equipment that may be used to treat the patient. For example, emergency medical personnel arriving on the scene may bring a second defibrillator, usually more full-featured than an AED. This second defibrillator may provide further therapy to the patient. Upon the patient's arrival at the hospital, the patient may be treated with the hospital's own defibrillator.

### SUMMARY

The invention provides techniques for communication between medical devices, such as defibrillators, that are used to monitor or treat a patient. The devices may engage in one-way or two-way communication, using the electrical conduction of the body of the patient as a transmission medium to carry information. In an example of one-way communication, a first medical device detects the presence of a second medical device. In an example of two-way communication, the first medical device transmits data to the second medical device via the electrical conduction of the body of the patient.

In an exemplary application, the patient is initially treated with an AED. Later, when emergency medical personnel arrive, a second defibrillator is coupled to the body of the patient. The two defibrillators detect one another, establish communication, and relay information to one another through the body of the patient. The AED may, for example, transmit to the full-featured defibrillator data pertaining to the condition of the patient and data pertaining to defibrillation therapy administered by the AED. The second defibrillator may notify the emergency medical personnel of the received data, and may coordinate therapy with the therapy previously administered by the AED.

When the full-featured defibrillator is later taken to a hospital, the full-featured defibrillator may be coupled to a hospital device such as a defibrillator, a monitor or a recording device. The full-featured defibrillator transmits to the hospital device the data collected by the full-featured defibrillator, including the patient condition and treatment data received from the AED. In this manner, the data collected by the AED can be received by the hospital device, even though the hospital device is not put in direct contact with the AED. The hospital device merges the data from the devices to generate a complete record of the monitoring and treatment of the patient.

In one embodiment, the invention is directed to a method that may be performed by a device such a defibrillator. The method includes detecting a signal from a medical device via electrodes coupled to a body of a patient, establishing communication with the medical device, and transmitting information to the medical device by electrical conduction through the body of the patient. Information may be transmitted by modulating an excitation current using modulation techniques such as amplitude modulation, frequency modulation or phase modulation.

In another embodiment, the invention is directed to a method comprising establishing communication with a second medical device and receiving from the second medical device data collected by a first medical device. The second medical device received the data from the first medical device by electrical conduction through a body of a patient. The first and second medical devices may be devices such as defibrillators and patient monitors, and the data may pertain to the condition of the patient, treatment of the patient, or both. Data received in this way may be merged to generate a record of the monitoring and treatment of the patient.

In a further embodiment, the invention presents a device comprising a signal generator to generate an excitation current. The excitation current is modulated to encode data. The device also includes a controller to generate a modulation signal to modulate the excitation current and at least two electrodes for coupling to the body of a patient and for delivering the modulated excitation current to the body of the patient.

The invention can provide one or more advantages. For example, a first device, such as an external defibrillator, can detect the presence of a second device, such as an implanted pacemaker. Even if the two devices cannot communicate, one device may avoid interfering with the operation of the other. In addition, the presence of some medical devices can be more readily detected by electrical conduction through the body, and may not be as easily detected in other ways.

Furthermore, device-to-device communication may be rapid and may avoid problems with external sources of interference, such as crowded wireless frequencies.

Also, the techniques for merging data from multiple devices is advantageous, not only in treatment of the patient, but also in managing medical records and in reviewing the quality of treatments and health care protocols.

The details of one or more embodiments of the invention are set forth in the accompanying drawings and the description below. Other features, objects, and advantages of the invention will be apparent from the description and drawings, and from the claims.

### BRIEF DESCRIPTION OF DRAWINGS

FIG. 1 is a schematic diagram of an external defibrillator and a patient.

FIG. 2 is a block diagram illustrating an exemplary embodiment of a programmable impedance system.

FIG. 3 is a schematic diagram of an automated external defibrillator and a full-featured defibrillator coupled to the body of a patient.

FIG. 4 is a flow diagram showing techniques for communication between medical devices coupled to the body of a patient.

FIG. 5 is three parallel flow diagrams illustrating the interaction among a fist medical device, a second medical device and a hospital device.

### DETAILED DESCRIPTION

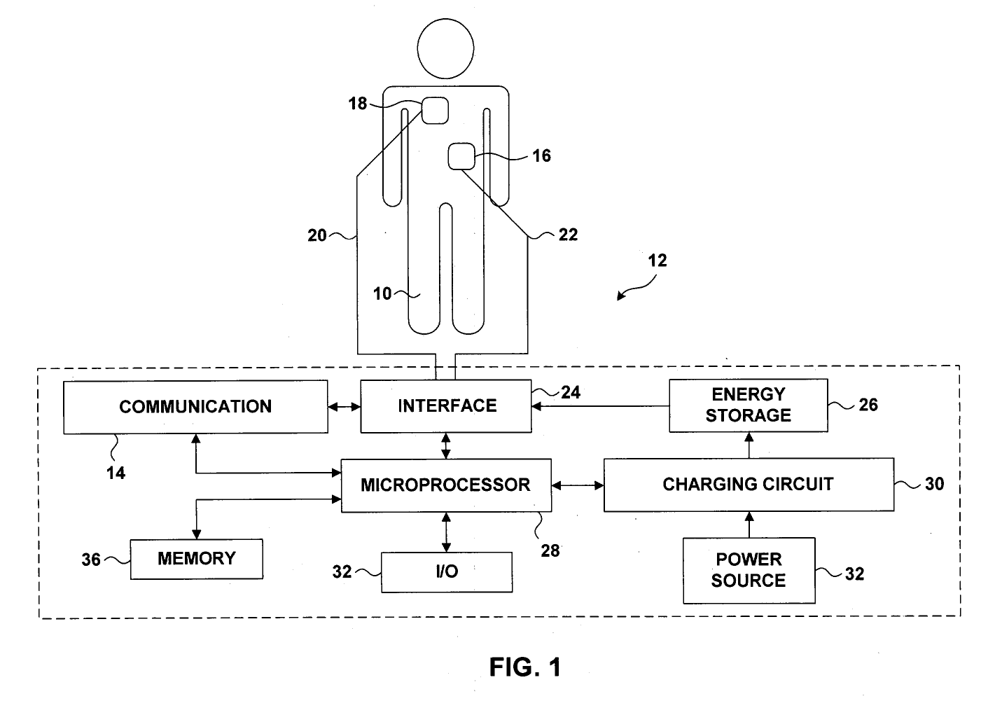

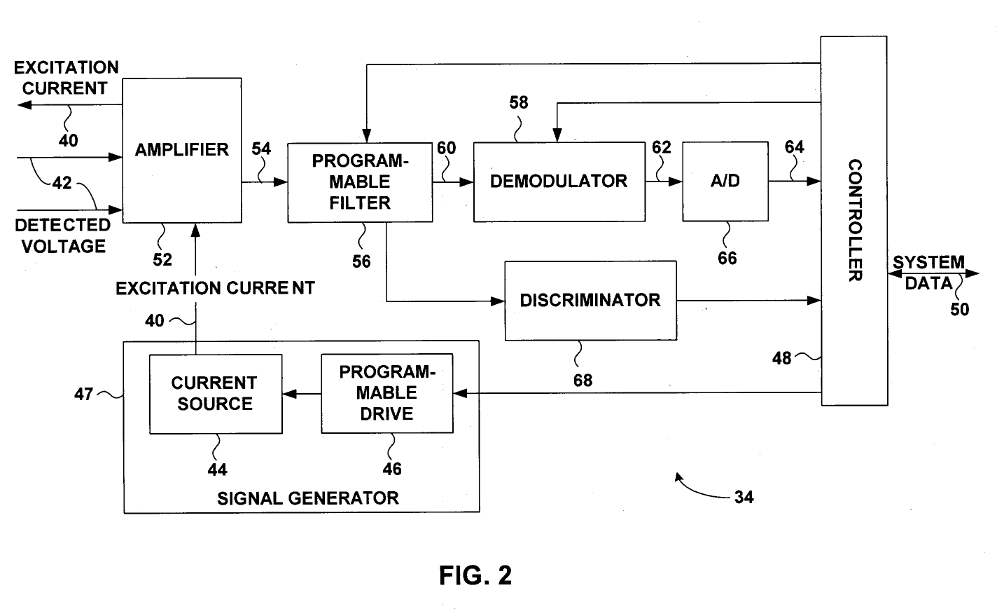

FIG. 1 is a block diagram showing a patient 10 coupled to an external defibrillator 12. In accordance with the invention, defibrillator 12 includes a communication module 14 for communication with one or more other devices, using electrical conduction through the body of patient 10 as a transmission medium. As will be described in more detail in connection with FIG. 2, communication module 14 includes a controllable current source for generating an excitation current, also called a “carrier,” and circuitry for modulating the excitation current to carry information. The modulated excitation current is applied to patient 10 through electrodes 16 and 18 to send information to other one or more other devices. Communication module 14 may also receive electrical signals from the other devices conducted through the body of patient 10 by measuring the voltage between electrodes 16 and 18.

The excitation current is an alternating current signal, and may be modulated by a modulating signal. Modulation may employ any modulation technique, such as amplitude modulation, phase modulation or frequency modulation. Data may be encoded in any fashion, such as by frequency-shift keying or quadrature amplitude modulation. Modulation of the excitation current allows information to be encoded in the excitation current.

In one embodiment of the invention, communication module 14 transmits a communication signal via the body of patient 10. The communication signal may convey information to a second medical device coupled to patient 10. The conveyed information may include an attention signal as described below, device data and patient data. Communication module 14 may also sense a communication signal originating from a second medical device via patient 10. Furthermore, communication module 14 may establish two-way communication with the second medical device, and may exchange information with the second medical device.

Electrodes 16 and 18 may be hand-held electrode paddles or adhesive electrode pads placed on the skin of patient 10. The body of patient 10 provides an electrical path between electrodes 16 and 18. Defibrillator 12 senses the electrical activity of the heart of patient 10 and administers defibrillation therapy to patient 10 via electrodes 16 and 18. Electrodes 16 and 18 are coupled to defibrillator 12 via conductors 20 and 22 and interface 24. In a typical application, interface 24 includes a receptacle, and connectors 20, 22 plug into the receptacle.

Interface 24 includes a switch (not shown in FIG. 1) that, when activated, couples an energy storage device 26 to electrodes 16 and 18. Energy storage device 26 stores the energy to be delivered to patient 10. The switch may be of conventional design and may be formed, for example, of electrically operated relays. Alternatively, the switch may comprise an arrangement of solid-state devices such as silicon-controlled rectifiers or insulated gate bipolar transistors.

Energy storage device 26 includes components, such one or more capacitors, that store the energy to be delivered to patient 10 via electrodes 16 and 18. Before a defibrillation pulse may be delivered to patient 10, energy storage device 26 must be charged. A microprocessor 28 directs a charging circuit 30 to charge energy storage device 26 to a high voltage level. Charging circuit 30 comprises, for example, a flyback charger that transfers energy from a power source 32 to energy storage device 26. Because the life of patient 10 may depend upon receiving defibrillation, charging should take place rapidly so that the defibrillation shock may be delivered with little delay.

When the energy stored in energy storage device 26 reaches the desired level, defibrillator 12 is ready to deliver the defibrillation shock. The shock may be delivered automatically or manually. When the shock is delivered automatically, microprocessor 28 activates an input/output (I/O) device 34, such as an indicator light or a voice prompt, that warns the operator that defibrillator 12 is ready to deliver a defibrillation shock to patient 10. The warning informs the operator of the impending shock so that no one other than patient 10 will receive the defibrillation shock. Microprocessor 28 then activates the switch to electrically connect energy storage device 26 to electrodes 16 and 18, and thereby deliver a defibrillation shock to patient 10. In the case of a manual delivery, microprocessor 28 may activate an I/O device 34 that informs the operator that defibrillator 12 is ready to deliver a defibrillation shock to patient 10. The operator may activate the switch by manual operation, such as pressing a button, and thereby deliver a defibrillation shock to patient 10.

Microprocessor 28 may also modulate the electrical pulse delivered to patient 10. Microprocessor 28 may, for example, regulate the shape of the waveform of the electrical pulse and the duration of the pulse.

Microprocessor 28 may perform other functions as well, such as monitoring electrocardiogram (ECG) signals sensed via electrodes 16 and 18 and received via interface 24. Microprocessor 28 may determine whether patient 10 suffers from a condition that requires a defibrillation shock, based upon the ECG signals. In addition, microprocessor 28 may also evaluate the efficacy of an administered defibrillation shock, determine whether an additional shock is warranted, and the magnitude of energy to be delivered in the additional shock.

The goal of defibrillation is to depolarize the heart with electrical current and cause the heart to reestablish a normal sinus rhythm. In some patients, one shock is insufficient to reestablish normal rhythm, and one or more additional defibrillation shocks may be required. Before another shock may be administered, however, charging circuit 30 ordinarily must transfer energy from power source 32 to energy storage device 26, thereby recharging energy storage device 26. In recharging energy storage device 26, as in the initial charging, time is of the essence, and charging circuit 30 therefore charges energy storage device 26 quickly. The energy to be delivered to patient 10 need not be the same in each shock.

Power source 32 may comprise, for example, batteries and/or an adapter to an exterior power source such as an electrical outlet. In addition to supplying energy to charging circuit 30 and energy storage device 26, power source 32 also supplies power to components such as microprocessor 28 and I/O device 34, e.g., via a power supply circuit (not shown in FIG. 1).

In one embodiment of the invention, communication module 14 delivers a communication signal via patient 10. The communication signal may convey information to a second medical device coupled to patient 10. The conveyed information may include device data and patient data The conveyed information may also include an attention signal that acts as a beacon. Communication module 14 may also sense a communication signal originating from a second medical device via patient 10. Furthermore, communication module 14 may establish two-way communication with the second medical device, and may exchange information with the second medical device.

Defibrillator 12 includes memory 36. Memory 36 stores instructions that direct the operation of microprocessor 28. In addition, memory 36 stores information about patient 10 and defibrillator 12. For example, memory 36 may store the ECG of patient 10, an analysis of the ECG, and whether a shock was indicated. Memory 36 may further store information about shocks delivered to patient 10, such as the number of shocks, the energy delivered per shock, the timing of shocks and the patient response to shocks. Memory 36 may include volatile storage, such as random access memory, and/or non-volatile storage, such as Flash memory or a hard disk.

When defibrillator 12 communicates with a second medical device as described below, defibrillator 12 may transmit information to the second medical device. In particular, information stored in memory 36 may be used by communication module 14 to generate a modulating signal that modulates the excitation current. In this way, communication module 14 may encode information in the excitation current. The excitation current may carry information such as the number of shocks delivered to patient 10 and the energy delivered per shock. Such information may be useful to a second defibrillator device that “takes over” responsibility for defibrillation therapy from defibrillator 12, e.g., upon arrival of paramedics.

FIG. 2 is a block diagram illustrating an exemplary embodiment of communication module 14. Communication module 14 supplies an excitation current 40 that is applied to patient 10. In addition, communication module 14 may sense a voltage 42 across electrodes 16 and 18 (not shown in FIG. 2).

Excitation current 40 is supplied by a controlled current source 44. A typical excitation current has a small current magnitude, such as 100 microamperes. Current source 44 is controlled by a programmable drive 46, which modulates excitation current 40. Current source 44 and programmable drive 46 cooperate as a signal generator 47. The invention encompasses other implementations of signal generator 47 as well. Signal generator 47 may encode data in excitation current 40 using amplitude modulation, phase modulation, frequency modulation, or any other modulation technique. When excitation current 40 is applied to patient 10, the data may be transmitted to a second medical device by electrical conduction through the body of patient 10.

Controller 48 supplies the information to be encoded in excitation current 40. In other words, controller 48 generates the modulating signal that modulates excitation current 40. The modulating signal may include system data 50 supplied to communication module 14. System data 50 comprises data such as data pertaining to the condition or treatment of patient 10. The modulating signal may also include information generated by controller 48.

Communication module 14 may receive input signals as voltage 42 between electrodes 16 and 18. The received signals may have been transmitted by a second medical device using electrical conduction through the body of patient 10 as a transmission medium. Voltage 42 is supplied to an amplifier 52, which finds the voltage difference 54 between electrodes 16 and 18. Amplifier 52 may also amplify the voltage difference and perform some filtering of noise from the input signal. Amplifier 52 is the gateway between communication module 14 and interface 24. Accordingly, excitation current 40 is channeled through amplifier 52. In addition, amplifier 52 may provide protection to communication module 14 from electrical surges.

Programmable filter 56 receives voltage signal 54. Programmable filter 56 may be, for example, a band pass filter with a variable center frequency. Controller 48 controls the selection of the center frequency and supplies the selected center frequency to programmable filter 56. Controller 48 may also control the bandwidth of programmable filter 56. A demodulator 58 receives filtered signal 60. Demodulator 58 recovers the signal or signals encoded in the voltage difference. Recovered signals 62 may be converted to digital signals 64 by analog-to-digital (A/D) converter 66 for processing by controller 48.

In this way, controller 48 regulates one-way or two-way communication between defibrillator 12 and a second medical device. Controller 48 receives and processes signals received via electrical conduction through the body of patient 10, and also transmits signals by electrical conduction through the body of patient 10.

Communication module 14 may further comprise a discriminator 68 that discriminates between a signal generated by a device and a biological signal, i.e., a signal generated by the body of patient 10. Discriminator 68 can detect whether a second device is present. In addition, discriminator 68 may be able to recognize the device that generates the signal. As will be discussed below, received signals may have distinguishing characteristics.

FIG. 2 shows an exemplary logical relationship among the components of communication module 14, but is not limited to any particular hardware or software implementation. For example, some components, such as programmable filter 56 and demodulator 58, may be realized as analog components, digital components, or a combination of analog and digital components. A/D converter 66 may be located so as to convert analog signals to digital signals where needed.

Furthermore, communication module 14 may include additional components that are not shown in FIG. 2, such as a band pass filter to shape and remove noise from excitation current 40. Programmable impedance system 14 may also exclude components that shown in FIG. 2. The functions of controller 48, for example, may be performed by microprocessor 28. Moreover, communication module 14 may include additional functionality, such as components for measuring the impedance of the body of patient 10.

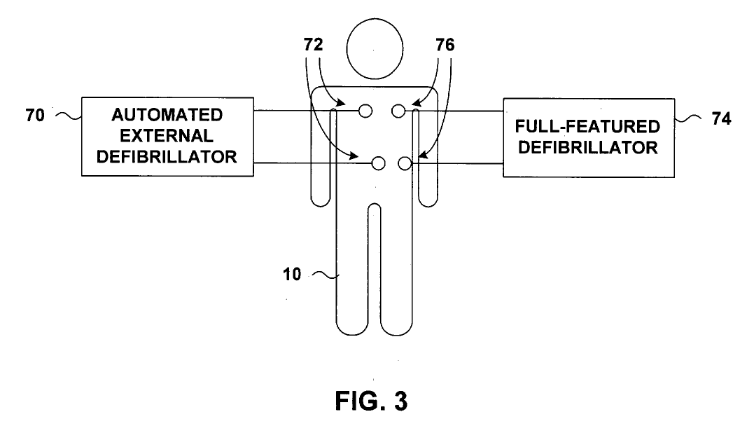

FIG. 3 demonstrates a scenario in which the invention may be implemented. Patient 10 collapses at a public venue such as an airport. A security guard arrives on the scene with an automated external defibrillator (AED) 70, and attaches the electrodes 72 of AED 70 to the chest of patient 10. AED 70 senses electrical impulses via electrodes 72, determines that patient 10 exhibits a shockable rhythm, and begins the process of delivering a shock. In particular, AED 70 stores energy to be delivered to patient 10 and delivers the shock automatically or manually. AED 70 then senses electrical impulses via electrodes 72 to evaluate the response of patient 10 to the shock. Based upon the response of patient 10, AED 70 may administer a second shock. The second shock may deliver a different quantity of energy than the first shock.

Paramedics arrive on the scene shortly, bringing a full-featured defibrillator 74. The paramedics attach electrodes 76 of full-featured defibrillator 74 to patient 10. While electrode sets 72 and 76 are attached to patient 10, defibrillators 70 and 74 detect the presence of one another and exchange information. In particular, AED 70 may transmit information to full-featured defibrillator 74 as to the rhythms exhibited by patient 10, the number of shocks administered, the timing of the shocks and the energy of the shocks.

The exchange of information between AED 70 and full-featured defibrillator 74 serves many functions. First, full-featured defibrillator 74 receives information concerning treatment provided and the efficacy of the treatment. AED 70 may have administered defibrillation shocks of 200 J and 300 J to patient 10, for example, without restoring normal sinus rhythm. When AED 70 communicates this information to full-featured defibrillator 74, full-featured defibrillator 74 may report the treatment history to the paramedics by a display screen or other input-output device. The paramedics may use this treatment history to determine whether additional shocks are indicated at the time and the energy to be delivered in the shocks. In addition, full-featured defibrillator 74 may automatically select a therapy as a function of the information received from AED 70. Full-featured defibrillator 74 may, for example, escalate the defibrillation therapy by bypassing administration of shocks of 200 J and 300 J and selecting a defibrillation therapy at a higher energy, such as 360 J.

In addition, the exchange of information may be used by full-featured defibrillator 74 to prepare an event log. An event log includes a history of events in the treatment of patient 10, such as the time of activation of AED 70, the time that the heart rhythm of patient 10 was analyzed, the time that a shock as administered, and the energy delivered in the shock. The event log may be later downloaded from full-featured defibrillator 74 to a device at a hospital, and may be merged with other records so that the hospital may have a complete “run report,” i.e., a complete record of the treatment of patient 10 including pre-hospital treatment.

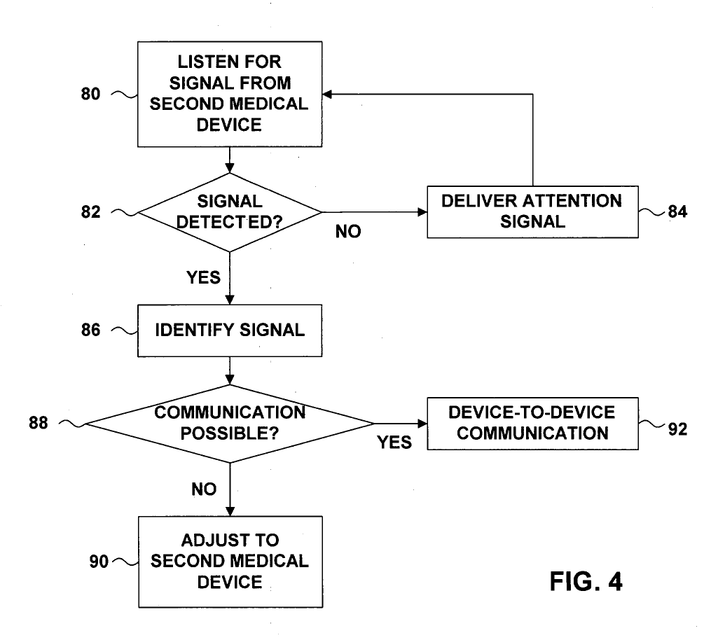

FIG. 4 is a flow diagram illustrating communication techniques that may be employed by a first medical device such as an AED in communication with a second medical device. The techniques shown in FIG. 4 may be in addition to or subordinate to other techniques performed by the first medical device. For example, AED 70 may assign a higher priority to reading the heart rhythm of patient 10 than to communicating with another medical device, and may suspend communication techniques while reading the heart rhythm.

Communication may begin when the electrodes of the first medical device are placed on the body of patient 10. The first medical device may listen for one or more signals that are generated by a possible second medical device (80). Listening may include suspending generation of excitation current 40, scanning a range of frequencies with programmable filter 56 and listening for device-generated signals with discriminator 68. Device-generated signals may be of many types.

A received signal may comprise, for example, a “lead off” signal. A lead off signal is an electrical signal employed by a medical device to determine whether the one or more leads, or electrodes, are properly connected to patient 10. The lead off signal, usually an alternating current signal of a regular frequency and a known low amperage is driven into patient 10, and the medical device driving the lead off signal senses the voltage generated across the leads in response to the lead off signal. When the medical device senses a large voltage, a high impedance is indicated, suggesting a poor connection with patient 10.

The first medical device may sense the presence of the second medical device by sensing the lead off signal generated by the second medical device. The lead off signal may be discriminated from a patient-generated biological signal by, for example, the regularity of the frequency of the signal.

The lead off signal is an example of a signal that is intentionally conducted through patient 10. Other medical devices may conduct signals unintentionally through patient 10. An example of an unintentional signal may be electromagnetic interference (EMI) generated by the circuitry of the second medical device.

In some patients, the first medical device may detect a signal from an implanted pacemaker or defibrillator. Implanted pacemakers and defibrillators generate voltage spikes that may be detected by the first medical device. Voltage spikes may be indicative of the presence of a medical device rather than biological activity.

Another signal that may be detected is an attention signal. An attention signal is a signal that acts as a beacon, and is difficult to mistake for a biological signal. A medical device uses an attention signal to announce its presence to other medical devices. If the first medical device does not detect other signals (82), the first medical device may generate an attention signal (84) that will notify a second medical device, such as a second medical device attached to patient 10 at a later time, of the presence of the first medical device.

If the first medical device does detect other signals (82), the first medical device may try to identify the signal (86) as an attention signal, a lead off signal, a voltage spike from an implanted device, or some other kind of signal. Some received signals, such as an attention signal, may indicate that device-to-device communication is possible, and some signals may indicate the presence of medical devices that lack device-to-device communication capability (88).

In circumstances in which device-to-device communication is not possible, there are advantages for the first medical device knowing of the presence of the second medical device. The first medical device may make adjustments to the therapy (90) because of the presence of the second medical device. If, for example, an external defibrillator detects the presence of an implanted defibrillator, the external defibrillator may avoid delivering a shock, as uncoordinated shocks administered by two defibrillators might be harmful to patient 10. The external defibrillator may also adjust its arrhythmia detection algorithms when the external defibrillator detects an implanted pacemaker or defibrillator, to improve the arrhythmia detection performance of the external defibrillator.

When device-to-device communication is possible, communication may be initiated (92) by, for example, paging the second medical device and establishing a communication protocol. Establishing a communication protocol may include, for example, exchanging identification data so that the medical devices can identify each other. In some cases, communication may be established, and data may be exchanged, in a very short time.

In some circumstances, device-to-device communication may be one-way communication. Some kinds of implanted devices, for example, may respond to command to suspend operation so that the first medical device can provide treatment to patient 10 without interference from the implanted second medical device.

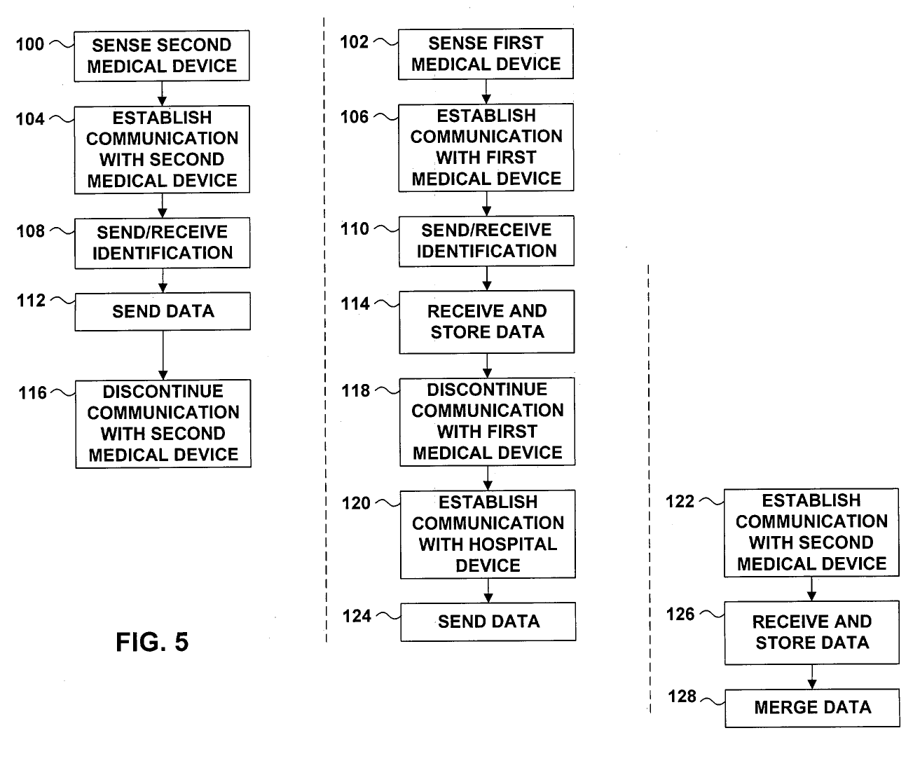

FIG. 5 illustrates communication operations that may occur among medical devices. The left flow diagram illustrates exemplary techniques employed by a first medical device, such as AED 70 in FIG. 3. The center flow diagram illustrates exemplary techniques employed by a second medical device, such as full-featured defibrillator 74. The right flow diagram illustrates exemplary techniques employed by a device at a hospital, such as a treatment device or a record-keeping computer.

When the first and second medical devices are coupled to patient 10 as depicted in FIG. 3, the devices sense one another (100, 102) and establish communication with one another (104, 106). The first and second medical device may identify themselves to one another (108, 110). Identification may include identifying the kind of device, such as defibrillator or a monitor. Identification may also comprise reporting a manufacturer and model. When the devices have identified themselves to one another, the devices may, for example, learn what information each device may have or require, learn what communication protocols are supported, and establish a direction of information flow.

In FIG. 5, the first medical device transmits information to the second medical device (112). The transmitted information may include, for example, an ECG of patient 10, an analysis of the ECG, the number of shocks administered, the energy delivered per shock, the timing of shocks and the patient response to shocks. The second medical device receives the data and stores the data in the memory of the second medical device (114). The second medical device also stores in memory other events in which second medical device participated, such as a new analysis of the ECG, the number of shocks administered by the second medical device, the energy delivered per shock, and the timing of shocks. The transmitted information may be detailed or abbreviated.

The transmitted information may also include other information, such as information pertaining to a course of treatment. For example, medical personnel may wish to use two defibrillators to deliver simultaneous shocks to a large bodied patient. The defibrillators may communicate with one another to coordinate the shocks to increase the benefit to the patient and to reduce the risk that each defibrillator's shocks may damage the other defibrillator.

Communication between the first and second medical devices may be discontinued (116, 118) following transmission of data. In a typical application involving an AED and a full-featured defibrillator, the AED may be disconnected from the patient, with monitoring and therapy provided by the full-featured defibrillator.

When patient 10 is transported to the hospital, the full-featured defibrillator may also be transported to the hospital. At the hospital, the full-featured defibrillator establishes communication with a hospital device, such as a defibrillator, a monitor or a recording device (120, 122). The full-featured defibrillator and the hospital device may communicate via any medium, including hard wired and wireless connections. The full-featured defibrillator and the hospital device may also communicate via electrical conduction through the body of patient 10.

The full-featured defibrillator sends to the hospital device the data collected from the first medical device, as well as data regarding events in which second medical device participated (124). The hospital device receives and stores the data (126). The hospital device also merges the data from the first medical device, the data from the second medical device, and other data received at the hospital concerning patient 10. In this way, the first medical device communicates with the hospital device through the second medical device, and the hospital device obtains a complete record of the monitoring and treatment of patient 10.

Merged data is advantageous for treatment of patient 10. The treating physician can learn about all monitoring and treatment performed before patient 10 arrived at the hospital, and can treat patient 10 accordingly. In addition, the hospital device assembles the data collected from multiple medical devices, relieving a records person of this task.

Merged data may also be advantageous to the health care system. As part of a quality assurance program, for example, a regulating authority may use to the data to assess whether patient 10 was treated according to established health care protocols, or whether the treatment was effective. The data may be of help in the development of more effective treatments and health care protocols.

Furthermore, because the information may be transmitted by electrical conduction through the body of the patient, the techniques of the invention provide additional security that the information will pertain to that particular patient. Device-to-device communication that uses wireless techniques, for example, may be prone to communication errors when two or more patients are treated in proximity to one another.

The invention can provide other advantages. Because medical devices can interfere with one another, learning of the presence of a second medical device is advantageous, even if it is not possible to communicate with the second medical device. For example, a defibrillator, upon detecting the presence of a device such as a pacemaker implanted in patient 10, may advise the operator of the presence of the implanted device via an input/output device, and may adjust therapy to cooperate with or avoid interfering with the implanted device. The presence of a second medical device may be revealed by a signal intentionally introduced into the body, such as a lead off signal, or a signal unintentionally introduced into the body, such as EMI. Signals of this kind may be readily detected by way of body conduction, and may be less likely to be detected by other methods.

In addition, conduction of signals from a first medical device to a second medical device via electrical conduction through the body may avoid some external sources of interference. Furthermore, information exchange using the body as a transmission medium can be rapid. The rapidity of information exchange may be enhanced by the fact that communication may begin when the devices are coupled to the body, and it is not necessary to take the time to couple the devices together with a dedicated connector.

Various embodiments of the invention have been described. These embodiments are illustrative of the practice of the invention. Various modifications may be made without departing from the scope of the claims. For example, the techniques described above may be embodied as a medium that stores one or more instructions to cause a digital processor to implement the techniques. Such storage medium may include, for example, non-volatile RAM, Flash memory, read-only memory, or magnetic or optical storage media.

In addition, although the invention may be used with defibrillators, the invention is not limited to use with defibrillators. A non-treating device such as a patient monitor may communicate with a second device through the body of the patient. The non-treating device may transmit information about the condition of the patient, such as heart rate, blood pressure and temperature. In some circumstances, the non-treating device need not transmit any data pertaining to treatment.

The invention may also be practiced with treating devices other than defibrillators. A drug delivery system, for example, may communicate with a second medical device through the body of the patient. The drug delivery system may report what medicines have been administered and the timing and dosages of the administrations. These and other embodiments are within the scope of the following claims.

1. A method comprising:
detecting a signal from a medical device via electrodes coupled to a body of a patient; and
transmitting information to the medical device by electrical conduction through the body of the patient.

2. The method of claim 1, wherein the medical device is a defibrillator.

3. The method of claim 1, wherein transmitting information to the medical device comprises transmitting information about therapy provided to the patient.

4. The method of claim 1, wherein transmitting information to the medical device comprises transmitting a command to the medical device to suspend operation.

5. The method of claim 1, wherein transmitting information to the medical device comprises transmitting at least one of a kind of medical device, a manufacturer and a model.

6. The method of claim 1, wherein transmitting information to the medical device comprises modulating an excitation current with a modulating signal and applying the modulated excitation current to the body of the patient.

7. The method of claim 6, wherein modulating an excitation current with a modulating signal comprises modulating at least one of the amplitude, phase and frequency of the excitation current.

8. The method of claim 1, further comprising receiving information from the medical device by electrical conduction through the body of the patient.

9. A medium comprising one or more instructions to cause a processor to:
detect a signal from a medical device via electrodes coupled to a body of a patient; and
transmit information to the medical device by electrical conduction through the body of the patient.

10. The medium of claim 9, wherein the medical device is a defibrillator.

11. The medium of claim 9, wherein the instructions cause the processor to transmit information to the medical device by transmitting information about therapy provided to the patient.

12. The medium of claim 9, wherein the instructions cause the processor to transmit information to the medical device by transmitting a command to the medical device to suspend operation.

13. The medium of claim 9, wherein the instructions cause the processor to transmit information to the medical device by transmitting at least one of a kind of medical device, a manufacturer and a model.

14. The medium of claim 9, wherein the instructions cause the processor to transmit information to the medical device by modulating an excitation current with a modulating signal and applying the modulated excitation current to the body of the patient.

15. The medium of claim 14, wherein the instructions cause the processor to modulate an excitation current with a modulating signal by modulating at least one of the amplitude, phase and frequency of the excitation current.

16. The medium of claim 9, the instructions further causing the processor to receive information from the medical device by electrical conduction through the body of the patient.

17. A method comprising:
establishing communication with a second medical device; and
receiving from the second medical device data collected by a first medical device, the data having been received by the second medical device from the first medical device by electrical conduction through a body of a patient.

18. The method of claim 17, further comprising receiving from the second medical device data collected by the second medical device.

19. The method of claim 18, further comprising merging the data collected by the first medical device and the data collected by the second medical device.

20. The method of claim 17, wherein data collected by the first medical device comprises data pertaining to the condition of the patient monitored by the first medical device.

21. The method of claim 17, wherein data collected by the first medical device comprises data pertaining to the treatment of the patient administered by the first medical device.

22. A medium comprising one or more instructions to cause a processor to:
establish communication with a second medical device; and
receive from the second medical device data collected by a first medical device, the data having been received by the second medical device from the first medical device by electrical conduction through a body of a patient.

23. The medium of claim 22, the instructions further causing the processor to receive from the second medical device data collected by the second medical device.

24. The medium of claim 23, the instructions further causing the processor to merge the data collected by the first medical device and the data collected by the second medical device.

25. The medium of claim 22, wherein data collected by the first medical device comprises data pertaining to the condition of the patient monitored by the first medical device.

26. The medium of claim 22, wherein data collected by the first medical device comprises data pertaining to the treatment of the patient administered by the first medical device.

27. A device comprising:
a signal generator that transmits a data modulated signal; and
at least two electrodes for coupling to the body of a patient and for delivering the data modulated signal to the body of the patient.

28. The device of claim 27, further comprising a programmable filter to filter a received electrical signal conducted through the body of the patient, wherein the controller controls at least one of a center frequency and a bandwidth of the filter.

29. The device of claim 28, further comprising a demodulator to demodulate the filtered received electrical signal.

30. The device of claim 28, further comprising a discriminator to distinguish a signal generated by a second device and a signal generated by the patient.

31. The device of claim 27, wherein the device is a defibrillator.

32. The device of claim 27, wherein the device is a patient monitor.

33. The device of claim 27, further comprising a controller to generate a modulation signal to modulate the modulated signal.

34. A method comprising:
detecting a signal from a medical device via electrodes coupled to a body of a patient; and
selecting a therapy as a function of the signal received from the medical device.

35. The method of claim 34, wherein the signal comprises information about therapy provided to the patient by the medical device.

36. The method of claim 35, wherein the information comprises at least one of an electrocardiogram of the patient, an analysis of the electrocardiogram, information about the number of shocks delivered to the patient, information about the energy delivered in a shock, information about the timing of a plurality of shocks and information about the response of the patient to a shock.

37. The method of claim 34, wherein selecting a therapy comprises selecting an energy level for defibrillation therapy.

38. A medium comprising one or more instructions to cause a processor to:
detect a signal from a medical device via electrodes coupled to a body of a patient; and
select a therapy as a function of the signal received from the medical device.

39. The medium of claim 38, wherein the signal comprises information about therapy provided to the patient by the medical device.

40. The medium of claim 39, wherein the information comprises at least one of an electrocardiogram of the patient, an analysis of the electrocardiogram, information about the number of shocks delivered to the patient, information about the energy delivered in a shock, information about the timing of a plurality of shocks and information about the response of the patient to a shock.

41. The medium of claim 38, wherein the instructions cause the processor to select a therapy by selecting an energy level for defibrillation therapy.
